(19) United States
(12) Patent Application Publication
(10) Pub. No.: US 2005/0036953 A1
(43) Pub. Date: Feb. 17, 2005

(54) Topical compositions of ammonium lactate

(76) Inventors: Moshe Arkin, Kfar Shmaryahu (IL); Nir Avram, Meitar (IL); Olga Buriakovsky, Beer Sheva (IL); Amira Zeevi, Omer (IL); Benjamin Schneider, Rehovot (IL); Stephen Cherkez, Caesarea (IL); Stella Nivy, Tel Aviv (IL)

Correspondence Address: Martin MOYNIHAN;c/o ANTHONY CASTORINA, SUITE 207, 2001 JEFFERSON DAVIS HIGHWAY, ARLINGTON, VA, 22202, US

(21) Appl. No.: 10/850,462

(22) Filed: May 21, 2004

Related U.S. Application Data
(60) Provisional application No. 60/494,579, filed on Aug. 13, 2003.
(60) Provisional application No. 60/494,581, filed on Aug. 13, 2003.
(60) Provisional application No. 60/510,554, filed on Oct. 14, 2003.
(60) Provisional application No. 60/527,279, filed on Dec. 8, 2003.

(51) Int. Cl. A61K 9/00; A61K 31/19

(52) U.S. Cl. 424/47; 514/557

(57) ABSTRACT

Pharmaceutical, cosmetic and cosmeceutical compositions for topical application, containing, as an active ingredient, ammonium lactate and/or any other alpha-hydroxy carboxylic acid and/or salts thereof, processes of manufacturing these compositions and uses of these compositions in the treatment of medical and cosmetic conditions such as conditions associated with dry skin or scalp.

This application claims the benefit of priority from U.S. Provisional Patent Application Nos. 60/494,579, 60/494,581, filed Aug. 13, 2003, U.S. Provisional Patent Application No. 60/510,554, filed Oct. 14, 2003 and U.S. Provisional Patent Application No. 60/527,279, filed Dec. 8, 2003, the teachings of which are hereby incorporated by reference in their entirety.

### FIELD AND BACKGROUND OF THE INVENTION

The present invention relates to pharmaceutical, cosmetic and cosmeceutic compositions for topical application, and their use in the treatment of medical, cosmetic and cosmeceutical conditions such as dry skin and/or scalp. Dry skin is a common condition associated with a plurality of disorders and frequently requires therapeutic intervention.

Dermatologists often call dry skin in later life “xerosis” or “ichthyosis” Xerosis is a term used to describe abnormal skin dryness. Ichthyosis is a term used to describe a group of cutaneous disorders characterized by increased or aberrant keratinisation, and resulting in non-inflammatory scaling of the skin. There are at least twenty varieties of ichthyosis, including inherited and acquired forms. Further details regarding xerosis and ichthyosis can be found in “Atlas of Clinical Dermatology” by Anthony du Vivier, 3rd edition (Jul. 17, 2002) Publisher: Churchill Livingstone, which is incorporated herein by reference.

Dry skin often leads to dermatitis, a condition in which the skin becomes red and itchy, and which is typically characterized by a crazy-paving appearance on the lower legs (eczema craquelé) or round patches scattered over the trunk and limbs (a dry form of nummular dermatitis). In some cases of dermatitis, such as, for example, winter itch, 7th age itch, or senile pruritus, the dry skin is just itchy, without much of a rash.

Dry skin results from, or is aggravated by, low humidity, sunlight, abrasive clothing and/or a repeated use of soaps, detergents or other lipid solvents, and is further strongly influenced by factors such as age, race, genetics, climate and lifestyle.

Numerous humidifying topical preparations containing emollients and moisturizers have been used over the years in the treatment of dry skin and more acute dermatological disorders which exhibit dry skin symptoms, such as, for example, ichthyosis, psoriasis, actinic damage, eczema and the like.

As is known in the art, the terms “moisturizer” (to add moisture) and “emollient” (to soften) are interchangeable as they describe different effects of the same agents on the skin, as is further detailed hereinunder.

“Moisturizers” is a general term used to describe substances that exert two basic actions: humectants, which are introduced into the stratum corneum to increase its water holding capacity; and occlusives, which provide a layer of oil on the surface of the skin to slow water loss and thus increase the moisture content of the stratum corneum. Some moisturizers contain both occlusives and humectants.

“Emollients” is a general term used to describe substances that cover the surface of the stratum corneum so as to prevent moisture loss, thus resulting in the closure of microcracks and fissures and restoration of the natural epidermal barrier. (Marie Loden, Clinics in Dermatology, 21, 145-157, 2003).

Herein, the terms “moisturizer”, “humectant”, “emollient” and the term “hydrating agent” are used interchangeably.

As is well recognized in the art, the final form of a topical composition plays an important role in its efficacy and its usage convenience, particularly in cases where the composition is used to treat a skin condition associated with dry skin and/or scalp.

The challenge in topically applying a composition is to achieve percutaneous penetration of the active agent to the site of treatment, in many cases the epidermis. At the same time, it is important that the composition should have desirable characteristics. Hence, application should be easy, smooth and should result in no irritation, discomfort or inconvenience. Desirably, the composition should not leave a residue on the surface of the skin.

Topical compositions in forms such as gels, ointments, lotions, creams, pads and pastes are often very viscous, requiring substantial rubbing to achieve penetration of the active agent to the affected skin layer, an act which often results in discomfort and further irritation. Non-viscous creams and lotions require quick and dexterous application as they are inclined to flow off the site of treatment before penetration of the active ingredient is achieved.

Contrary to the above, foams are well suited for the topical application of compositions. Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system.

A foam composition has physical characteristics which are dependent, at least in part, upon the choice and relative amounts of components such as solvents, propellants and surfactants, which may be present in the composition. The combination of these components determines the stability of the foam, which may retain its foam-like structure upon application or, alternatively, may be “a slow-breaking foam” or “a quick-breaking foam”, whereby this terminology relates to the behavior of the foam towards shearing action as is sustained when the foam is rubbed into or spread over a surface onto which it has been dispensed.

Many of the physical characteristics of foam compositions render it highly beneficial and advantageous over other forms. One such exemplary characteristic is the semi-solid to solid nature of the foam matrix, which allows the composition to be applied with the hand in any orientation without the risk of run off. Another beneficial characteristic of foams is their convenient application to large areas of the body surface. Furthermore, although foams can be water-based or hydroalcoholic, typically they are formulated with high alcohol content which, upon application to the skin of a user, quickly evaporates, driving the active ingredient through the upper skin layers to the site of treatment.

Ammonium lactate, which is the ammonium salt of lactic acid (also known as alpha-hydroxy propanoic acid), is a well-known emollient. Presently available commercial preparations that contain ammonium lactate include, for example, creams or lotions, which comprise up to 12% ammonium lactate (e.g., Lac-Hydrin 12% and Lac-Hydrin 5%, marketed by Westwood Squibb, a division of Bristol-Myers Squibb).

However, as is discussed hereinabove, these commercial preparations are disadvantageous as they are characterized by low absorption and high stickiness.

In view of its high moisturizing performance and the disadvantages associated with the presently available ammonium lactate-containing preparations, the present inventors have envisioned that a topical composition, formulated as a foam, which comprises ammonium lactate as an active ingredient, would be a highly potent composition for treating dry skin and scalp conditions and associated disorders.

Other formulations that include ammonium lactate have been described in the art.

For example, U.S. Pat. Nos. 3,879,537, 3,920,835, 3,984,470, 3,988,470, 4,021,572, 4,105,783, 4,246,261, 4,363,815, 5,422,370, and 5,554,597, all to Yu and Van Scott, disclose the use of α-hydroxy acids, α-keto acids and/or derivatives thereof, alone or in a combination with other active ingredients, in the treatment of various skin disease conditions such as dry skin, ichthyosis, eczema, palmar and plantar hyper-keratoses, dandruff, acne and warts. These patents specifically focus on low molecular weight α-hydroxy acids, α-keto acids and/or derivatives thereof, such as lactic acid and glycolic acid. The acids, according to the teachings of these patents, can be used as free acids or as the ammonium salts thereof, with the latter being preferred. Nevertheless, the compositions containing these active ingredients, according to these patents, are preferably formulated as lotions, creams, ointments or gels, which, as is described hereinabove, are disadvantageous in treating dry skin conditions. Moreover, these patents are completely silent with respect to foamable compositions that include α-hydroxy acids, α-keto acids and/or derivatives thereof.

U.S. Pat. No. 5,393,526 (to Castro) discloses a cosmetic facial foundation composition, which includes, as active ingredients, alpha-hydroxy carboxylate compound such as glycolic acid, lactic acid and/or their salts, in a concentration of from about 0.01% to about 10% by weight, and a rosmarinic acid or a salt thereof, in a concentration of from about 0.05% to about 5% by weight. This patent is directed to compositions that include a combination of these active ingredients, and therefore fails to teach a composition that includes ammonium lactate as the sole active ingredient. Furthermore, while according to the teachings of this patent such compositions are preferably formulated as liquids, gels, creams, lotions and powders, this patent is completely silent with respect to foamable compositions that include alpha-hydroxy acids and/or derivatives thereof.

U.S. Pat. No. 5,482,710 (to Slavtcheff) discloses a cosmetic composition for the treatment of pimples, blemishes and redness. The disclosed composition contains a keratolytic agent such as alpha-hydroxy carboxylic acid and/or salts thereof, in a concentration of from about 0.1% to about 10% by weight, and preferably from about 0.2% to about 1% by weight, and an anti-irritancy agent, which comprises a combination of water soluble anti-irritant and a water insoluble anti-irritant, in a concentration of from about 0.0001% to about 5% by weight, preferably from about 0.001% to about 1% by weight. Although this reference indicates that the disclosed composition can be formulated into various forms, preferred forms according to the teachings of this patent are a quick-drying gel or paste that forms a peelable facial mask. Hence, this patent is directed to compositions for treating external symptoms such as pimples, blemishes and redness and is therefore not directed to compositions that are aimed at being efficiently absorbed in the skin. Furthermore, this patent is directed to compositions that include a combination of the active ingredients described above, where the concentration of the keratolytic agent is relatively low and therefore fails to teach a composition that includes ammonium lactate as an active ingredient for treating dry skin and/or scalp conditions and associated disorders.

U.S. Pat. No. 5,679,324 (to Procter & Gamble Co.) discloses quick-breaking foamable fragrance compositions, which comprise a surfactant, a propellant, a fragrance and a thickener. These compositions, according to the teachings of this patent, may optionally further include skin moisturizers such as lactic acid and salts thereof. As this patent is directed to fragrance compositions, it fails to teach compositions for treating dry skin conditions, in which ammonium lactate is present in an effective amount for treating these conditions.

U.S. Pat. No. 6,086,903 (to Proctor & Gamble Company) discloses a personal treatment composition that comprises an enduring perfume composition. According to the teachings of this patent, ammonium lactate may optionally be added to the composition as a moisturizer, in a concentration of, as arbitrarily stated, between 0.1% and 20%, preferably, as stated, in a low concentration of between 2% and 5%. Again, this reference is directed to personal treatment compositions in which ammonium lactate is an optional adjuvant and fails to teach compositions for treating dry skin conditions, in which ammonium lactate is present in an effective amount for treating these conditions.

U.S. Patent Application No. 20020151446 (to Playtex Products, Inc.) discloses a foaming cleanser composition that comprises a mild surfactant system, a moisturizer system and a solvent system. According to the teachings of this patent, ammonium lactate can be one of the usable surfactants, and one of the suitable moisturizers. While this patent is directed to cleansing and conditioning of hair and skin, it fails to teach compositions for treating diseased or compromised skin.

Thus, the prior art fails to teach foamable compositions for treating dry skin and scalp conditions and associated disorders, which include ammonium lactate or any other alpha-hydroxy acid or salts thereof in an effective amount.

As the presently available ammonium lactate compositions are highly disadvantageous, particularly in treating dry skin and scalp conditions, and further as foamable compositions are highly advantageous in this respect, there is a widely recognized need for, and it would be highly advantageous to have, foamable compositions of ammonium lactate or analogs thereof for treating dry skin and scalp conditions and related disorders, as well as other medical, cosmetic and cosmeceutical conditions, devoid of the above limitations.

### SUMMARY OF THE INVENTION

The present inventors have now surprisingly found that foamable compositions that comprise an alpha-hydroxy carboxylic acid and/or a salt thereof, such as ammonium lactate, in a relatively high concentration, can serve as efficient pharmaceutical, cosmetic and cosmeceutical compositions for the treatment of various dermatological disorders (e.g., dry skin and/or scalp).

Hence, according to one aspect of the present invention there is provided a foamable pharmaceutical, cosmetic or cosmeceutical composition for topical application, which is identified for use in the treatment of a medical, cosmetic and/or cosmeceutical condition associated with dry skin and/or scalp. The composition of the present invention comprises one or more alpha-hydroxy carboxylic acids and/or salts thereof, one or more propellant(s) and a pharmaceutically, cosmetically or cosmeceutically acceptable carrier.

Each of the alpha-hydroxy carboxylic acids and/or the salts thereof according to the present invention preferably has a general formula:
RaRbC₁(OH)C₂(═O)OX
wherein, X is hydrogen, alkyl, cycloalkyl, aryl, halide or an ammonium ion, such that when X is an ammonium ion, O is negatively charged, or, alternatively, X is a C2-C10 alkyl being attached to C₂; and Ra and Rb are each independently hydrogen, halide, alkyl, alkenyl, alkynyl, cycloalkyl or aryl, or a salt thereof.

Preferably, the alpha-hydroxy carboxylic acids and/or the salts thereof comprise lactic acid and/or a salt thereof, e.g., ammonium lactate.

Hence, according to another aspect of the present invention there is provided a foamable pharmaceutical, cosmetic or cosmeceutical composition for topical application as described hereinabove, which comprises ammonium lactate, one or more propellant(s) and a pharmaceutically, cosmetically or cosmeceutically acceptable carrier.

According to further features in preferred embodiments of the invention described below, each of the foamable pharmaceutical, cosmetic or cosmeceutical compositions of the present invention is packaged in a packaging material and identified in print, in or on the packaging material, for use in the treatment of a medical, cosmetic and/or cosmeceutical condition associated with dry skin and/or scalp, such as, but not limited to, xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, and xeroderma pigmentosum.

According to still further features in the described preferred embodiments the concentration of the ammonium lactate or of the alpha-hydroxy carboxylic acid and/or the salt thereof is greater than 5 weight percentages of the total weight of the composition. More preferably it is greater than 10 weight percentages.

According to still further features in the described preferred embodiments the concentration of the ammonium lactate or of the alpha-hydroxy carboxylic acid and/or the salt thereof ranges between about 5.1 weight percentages and about 30 weight percentages of the composition. More preferably, it ranges between about 8 weight percentages and about 20 weight percentages of the composition.

The concentration of the propellant(s) preferably ranges between about 0.5 weight percentage and about 60 weight percentages, more preferably between about 10 weight percentages and about 20 weight percentages.

According to still further features in the described preferred embodiments each of the compositions of the present invention further comprises one or more additional active ingredient(s) selected from the group consisting of an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an antihistamine, a vitamin, a hormone and an antidandruff agent.

Such a composition can be further identified for use in the treatment of a condition in which applying this additional active agent is beneficial.

According to still further features in the described preferred embodiments each of the compositions of the present invention further comprises one or more ingredient(s) selected from the group consisting of a humectant, a deodorant agent, an antiperspirant, a sun screening agent, a sunless tanning agent, a hair conditioning agent, a pH adjusting agent, a chelating agent, a preservative, an emulsifier, an occlusive agent, an emollient, a thickener, a solubilizing agent, a penetration enhancer, an anti-irritant, a colorant and a surfactant. Preferably, the anti-irritant is either a water-soluble anti-irritant or a water-insoluble anti-irritant.

The pharmaceutical, cosmetic or cosmeceutical compositions of the present invention preferably have a pH value that ranges between about 4.0 and about 7.0, more preferably between about 5.0 and about 6.0.

The pharmaceutical, cosmetic or cosmeceutical compositions of the present invention are preferably devoid of an enduring perfume composition.

According to yet another aspect of the present invention there are provided processes of preparing the pharmaceutical, cosmetic or cosmeceutical compositions of the present invention. Each of the processes comprises admixing the ammonium lactate or the alpha-hydroxy carboxylic acid and/or the salt thereof, described hereinabove, the propellant(s) and a pharmaceutically, cosmetically or cosmeceutically acceptable carrier.

According to further features in preferred embodiments of the invention described below, in cases where the composition further comprises any of the active ingredients or ingredients described hereinabove, the processes further comprise admixing the active ingredient or any other ingredient with the ammonium lactate or the alpha-hydroxy carboxylic acid and/or the salt thereof, the propellant and the carrier.

According to still another aspect of the present invention there are provided methods of treating a medical, cosmetic and/or a cosmeceutical condition associated with dry skin and/or scalp and, optionally, other medical, cosmetic and/or cosmeceutical conditions. The methods comprise topically applying onto one or more biological surface(s) of a subject in need thereof, a pharmaceutically, cosmetically or cosmeceutically effective amount of any one of the compositions described hereinabove.

According to still further features in the described preferred embodiments the one or more biological surface(s) is selected from the group consisting of a lateral aspect of a forearm, a lateral aspect of a leg, an elbow, a palm, a foot, a backhand, a back and a scalp.

The present invention successfully addresses the shortcomings of the presently known configurations by providing foamable compositions containing ammonium lactate or any other alpha-hydroxy carboxylic acid and/or a salt thereof, which are highly efficient and convenient to use, and methods of treating conditions associated with dry skin and/or scalp, as well as other medical, cosmetic and cosmeceutical conditions, utilizing same.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a bar graph presenting the results obtained in a comparative test of the therapeutic effects of a foamable composition according to the present invention and of a control commercially available ammonium lactate cream composition.

### DESCRIPTION OF THE PREFERRED EMBODIMENTS

The present invention is of compositions for topical application which can be efficiently used in the treatment of various medical, cosmetic and/or cosmeceutical conditions. Specifically, the present invention is of (i) compositions for topical application, which contain, as an active ingredient, an alpha-hydroxy carboxylic acid and/or a salt thereof (e.g., ammonium lactate); (ii) processes of preparing these compositions; and (iii) their use in treating medical, cosmetic and/or cosmeceutical conditions such as, but not limited to, xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruff and xeroderma pigmentosum, as well as other dermatological conditions.

The principles and operation of the compositions, processes and methods according to the present invention may be better understood with reference to the Examples and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As is discussed in detail hereinabove, ammonium lactate is known as an efficient hydrating agent, which may therefore serve as a potent agent for treating dry skin conditions. The hydrating efficacy and hence, the therapeutic or cosmetic performance, as well as the usage convenience of hydrating agents and any other agents for topical application depend, inter alia, on the final form of the composition in which these agents are formulated.

As is further discussed in detail in the Background section above, foam formulations are well suited for the topical application of compositions. However, the presently known preparations that include a hydrating agent such as ammonium lactate as the active ingredient are typically formulated as creams, lotions, gels, ointment and the like, and therefore their efficacy and usage convenience are limited. On the other hand, although foam compositions which include ammonium lactate as an optional ingredient are known, these compositions are not aimed at treating dry skin or scalp conditions and therefore do not include ammonium lactate in a substantial concentration, such that it may serve as the main active hydrating agent.

In a search for an efficient composition for treating dry skin and scalp, as well as other medical, cosmetic and cosmeceutical disorders, which would overcome the disadvantages of the presently known formulations, the present inventors have surprisingly found that a composition that comprises ammonium lactate, preferably in a relatively high concentration, which is formulated as a foam, is highly efficient in the treatment of dry skin and/or scalp and is further characterized by improved absorption, after feel and comfort, as compared with the presently known formulations, and is devoid of stickiness and other adverse effects that accompany the use of the presently known formulations. The clinical efficacy and the improved and convenient application of the composition of the present invention are demonstrated in the Examples section that follows.

Hence, according to one aspect of the present invention, there is provided a foamable pharmaceutical, cosmetic or cosmeceutical composition for topical application, which is identified for use in the treatment of a medical, cosmetic and/or cosmeceutical condition associated with dry skin and/or scalp. The composition, according to this aspect of the present invention, comprises ammonium lactate, one or more propellant(s) and a pharmaceutically, cosmetically or cosmeceutically acceptable carrier.

As used herein, the phrase “topical application” describes application onto a biological surface, e.g., skin or scalp. Hence, the phrase “a composition for topical application” describes a composition that is applied to a subject by direct laying or spreading on the skin, scalp or any other biological surface of the subject.

As the compositions of the present invention are aimed at treating dry skin and/or scalp conditions, the topical application is preferably performed onto a dry skin area. The dry skin area can be, for example, the lateral aspect of a forearm, the lateral aspect of a leg, an elbow, a palm, a foot, a backhand, a back and/or a scalp.

As used herein throughout the term “comprising” means that other steps and ingredients which do not affect the end results can be added. This term encompasses the terms “consisting of” and “consisting essentially of”.

The phrase “consisting essentially of” means that the composition may include additional ingredients, but only if the additional ingredients do not materially alter the basic and novel characteristics of the claimed compositions or methods.

The phrase “active ingredient” as used herein means an ingredient that exerts a pharmaceutical, cosmetic or cosmeceutical activity. As ammonium lactate and other alpha-hydroxy acids are known hydrating agents and thus the composition of the present invention is preferably directed to treat or prevent dry skin or scalp, the phrase “active ingredient” whenever used herein in the context of ammonium lactate and other alpha-hydroxy acids refers to an ingredient that exerts hydration activity, namely, a hydrating agent. As ammonium lactate serves as the main active ingredient in the composition of the present invention, its concentration is relatively high, so as to efficiently serve as a hydrating agent.

Thus, the concentration of ammonium lactate in the composition of the present invention is preferably greater than 5 weight percentages, more preferably greater than 6 weight percentages, more preferably greater than 7 weight percentages, more preferably greater than 8 weight percentages, more preferably greater than 9 weight percentages, more preferably greater than 10 weight percentages, more preferably greater than 11 weight percentages, and more preferably it is about 12 weight percentages, 13 weight percentages or 14 weight percentages of the total weight of the composition.

The phrase “greater than” as used herein throughout with respect to a numerical indication (e.g., a concentration) encompasses any number (integral or fractional) that is greater than the indicated number.

However, the concentration of ammonium lactate in the composition of the present invention can further preferably be greater than 14 weight percentages and up to about 30 weight percentages. Hence, the ammonium lactate concentration in the composition preferably ranges between, for example, 5.1 weight percentages and about 30 weight percentages, more preferably between about 8 and about 20 weight percentages, more preferably between about 8 and about 16 weight percentages, and even more preferably between about 10 and about 16 weight percentages, with a concentration that ranges between An ammonium lactate concentration that ranges between about 12 weight percentages and about 14 weight percentages is being the presently most preferred concentration.

As used herein throughout, the phrase “weight percentages” describes the weight percentages (of an ingredient) of the total weight of a composition containing same.

As used herein the term “about” refers to ±10%.

Although ammonium lactate is a preferable hydrating agent, other hydrating agents that belong to the well-known alpha-hydroxy acids family, can be used as active ingredients in the foamable compositions of the present invention. Such hydrating agents which are used for treating dry skin are described, for example, in U.S. Pat. Nos. 3,879,537, 3,920,835, 3,984,470, 3,988,470, 4,021,572, 4,105,783, 4,246,261, 4,363,815, 5,422,370, and 5,554,597.

Hence, according to another aspect of the present invention, there is provided a pharmaceutical, cosmetic or cosmeceutical composition for topical application, which is identified for use in the treatment of a medical, cosmetic and/or cosmeceutical condition associated with dry skin and/or scalp. The composition, according to this aspect of the present invention, comprises, as an active ingredient, one or more alpha-hydroxy carboxylic acid(s) and/or salt(s) thereof, one or more propellants, and a pharmaceutically, cosmetically or cosmeceutically acceptable carrier.

The alpha-hydroxy carboxylic acid or the salt thereof according to the present invention can be generally described by the general formula:
RaRbC₁(OH)C₂(═O)OX
wherein:
X is hydrogen, alkyl, cycloalkyl, aryl, halide, or an ammonium ion, such that when X is an ammonium ion, O is negatively charged, or, alternatively, X is a C2-C10 alkyl that is attached to C₂; and
Ra and Rb are each independently hydrogen, halide, alkyl, alkenyl, alkynyl, cycloalkyl or aryl,
or a salt thereof.

As used herein, the term “alkyl” refers to a saturated aliphatic hydrocarbon including straight chain and branched chain groups. Preferably, the alkyl group has 1 to 20 carbon atoms. Whenever a numerical range; e.g., “1-20”, is stated herein, it means that the group, in this case the alkyl group, may contain 1 carbon atom, 2 carbon atoms, 3 carbon atoms, etc., up to and including 20 carbon atoms. More preferably, it is a medium size alkyl having 1 to 10 carbon atoms. Most preferably, it is a lower alkyl having 1 to 4 carbon atoms. The alkyl group may be substituted or unsubstituted. When substituted, the substituent group can be, for example, cycloalkyl, aryl, heteroaryl, heteroalicyclic, hydroxy, alkoxy, aryloxy, thiohydroxy, thioalkoxy, thioaryloxy, cyano, halide, carbonyl, thiocarbonyl, nitro and/or amino.

The term “cycloalkyl” refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, aryl, heteroaryl, heteroalicyclic, hydroxy, alkoxy, aryloxy, thiohydroxy, thioalkoxy, thioaryloxy, cyano, halide, carbonyl, thiocarbonyl, nitro and/or amino.

The term “aryl” refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, alkyl, cycloalkyl, aryl, heteroaryl, heteroalicyclic, hydroxy, alkoxy, aryloxy, thiohydroxy, thioalkoxy, thioaryloxy, cyano, halide, carbonyl, thiocarbonyl, nitro and/or amino.

The term “halide” refers to a fluoride, chloride, bromide or iodide radical.

The term “heteroaryl” refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, cycloalkyl, aryl, heteroaryl, heteroalicyclic, hydroxy, alkoxy, aryloxy, thiohydroxy, thioalkoxy, thioaryloxy, cyano, halide, carbonyl, thiocarbonyl, nitro and/or amino.

The term “heteroalicyclic” refers to a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydro furane, tetrahydropyrane, morpholino and the like. The heteroalicyclic may be substituted or unsubstituted. When substituted, the substituent group can be, for example, alkyl, cycloalkyl, aryl, heteroaryl, heteroalicyclic, hydroxy, alkoxy, aryloxy, thiohydroxy, thioalkoxy, thioaryloxy, cyano, halide, carbonyl, thiocarbonyl, nitro and/or amino.

The term “hydroxy” refers to an —OH group.

The term “alkoxy” refers to both an —O-alkyl and an —O-cycloalkyl group, as defined herein.

The term “aryloxy” refers to both an —O-aryl and an —O-heteroaryl group, as defined herein.

A “thiohydroxy” group refers to an —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “carbonyl” group refers to a —C(═O)—R′ group, where R′ is hydrogen, alkyl, alkenyl, cycloalkyl, aryl, heteroaryl (bonded through a ring carbon) or heteroalicyclic (bonded through a ring carbon) as defined herein.

A “thiocarbonyl” group refers to a —C(═S)—R″ group, where R″ is as defined herein for R′.

The term “amine” or “amino” refers to an —NR′R″ group where R′ and R″ are as defined herein.

The term “ammonium ion” refers to an —N⁺R′R″R′″ group where R′″ is as defined here for R′, and R′ and R″ are as defined herein.

A “nitro” group refers to an —NO₂ group.

A “cyano” group refers to a —C≡N group.

The term “alkenyl” refers to an alkyl group, as is defined hereinabove, which consists of at least two carbon atoms and at least one carbon-carbon double bond.

The term “alkynyl” refers to an alkyl group, as is defined hereinabove, which consists of at least two carbon atoms and at least one carbon-carbon triple bond.

Thus, the alpha-hydroxy carboxylic acid according to the present invention can be in a form of the free acid, such that X in the formula above is hydrogen, or a salt thereof, or, alternatively, the alpha-hydroxy carboxylic acid can be in a form of an ester, such that X in the formula above is alkyl, cycloalkyl or aryl, as these terms are defined hereinabove, or a salt thereof in some cases where the alkyl, cycloalkyl or aryl is substituted. The alpha-hydroxy carboxylic acid can further be in a form of an acyl chloride, such that X in the formula above is halide, or in a form of a lactone, such that X is the formula above is an alkyl that is attached to C₂, or a salt thereof, in some cases where the alkyl is substituted.

Further alternatively and preferably, the alpha-hydroxy carboxylic acid can be in a form of an ammonium salt, such that X in the formula hereinabove is an ammonium ion. As an ammonium ion is a positively charged ion, O in the formula above is negatively charged in this case.

Representative examples of alpha-hydroxy carboxylic acid and salts thereof that are usable in the context of this aspect of the present invention include, without limitation, 2-hydroxyethanoic acid (glycolic acid); 2-hydroxypropanoic acid (lactic acid); 2-methyl 2-hydroxypropanoic acid (methyllactic acid); 2-hydroxybutanoic acid; 2-hydroxypentanoic acid; 2-hydroxyhexanoic acid; 2-hydroxyheptanoic acid; 2-hydroxyoctanoic acid; 2-hydroxynonanoic acid; 2-hydroxydecanoic acid; 2-hydroxyundecanoic acid; 2-hydroxydodecanoic acid (alph.-hydroxylauric acid); 2-hydroxytetradecanoic acid (alpha-hydroxymyristic acid); 2-hydroxyhexadecanoic acid (alpha-hydroxypalmitic acid); 2-hydroxyoctadecanoic acid (alpha-hydroxystearic acid); 2-hydroxyeicosanoic acid (alpha-hydroxyarachidontc acid); 2-phenyl 2-hydroxyethanoic acid (mandelic acid); 2,2-diphenyl 2-hydroxyethanoic acid (benzilic acid); 3-phenyl 2-hydroxypropanoic acid (phenyllactic acid); 2-phenyl 2-methyl 2-hydroxyethanoic acid (atrolactic acid); 2-(4′-hydroxyphenyl) 2-hydroxyethanoic acid; 2-(4′-chlorophenyl 2-hydroxyethanoic acid; 2-(3′-hydroxy-4′-methoxyphenyl) 2-hydroxyethanoic acid; 2-(4′-hydroxy-3′-methoxyphenyl) 2-hydroxyethanoic acid; 3-(2-hydroxyphenyl) 2-hydroxypropanoic acid; 3-(4′-hydroxyphenyl) 2-hydroxypropanoic acid; 2-(3′,4′-dihydroxyphenyl) 2-hydroxyethanoic acid; and any salt thereof.

The concentration of the alpha-hydroxy carboxylic acid or the salt thereof in the composition according to this aspect of the present invention is greater than 5 weight percentages of the composition, preferably greater than 6 weight percentages, more preferably greater than 7 weight percentages, more preferably greater than 8 weight percentages, more preferably greater than 9 weight percentages, more preferably greater than 10 weight percentages, more preferably greater than 11 weight percentages, and most preferably is about 12 weight percentages, 13 weight percentages or 14 weight percentages.

The concentration of the alpha-hydroxy carboxylic acid or the salt thereof in the composition of the present invention can further preferably be greater than 14 weight percentages and up to 20 weight percentages. Hence, the concentration of the ammonium salt of the organic acid in the composition preferably ranges between 5.1 weight percentages and about 20 weight percentages, more preferably between about 8 and about 16 weight percentages, more preferably between about 10 and about 16 weight percentages, whereby a concentration that ranges between about 12 and about 14 weight percentages is being the presently most preferred.

Each of the compositions of the present invention, described hereinabove, further includes a pharmaceutically, cosmetically or cosmeceutically acceptable carrier.

As used herein, the term “pharmaceutically, cosmetically or cosmeceutically acceptable carrier” describes a carrier or a diluent that does not cause significant irritation to an organism and does not abrogate the biological activity and properties of the applied active ingredient.

Examples of acceptable carriers that are usable in the context of the present invention include carrier materials that are well-known for use in the cosmetic and medical arts as bases for foams, aerosols and the like.

The compositions of the present invention are formulated in the form of a foam. Preferably, the foam is formed by the passage of a pressurized mixture of a concentrate and a propellant through a nozzle. Preferably, the propellant is in the form of a compressed gas, typically a liquefiable gas. The mixture is preferably contained in a dispenser equipped with a dispensing head and valve, and pressurized with the propellant. Upon discharge of the composition through the dispensing head, the volatilization of the dispersed liquid droplets of propellant causes the dispensed concentrate to foam. Depending upon the precise formulation of the concentrate and the propellant, the dispensed product may range from a dense creamy foam to a light foam, dependent on desired aesthetics in the hand and when spread onto the substrate.

The concentration of the propellant in the composition preferably ranges between about 0.5 and about 60 weight percentages, more preferably between about 1 and about 20 weight percentages of the total composition.

Any propellant suitable for use in pharmaceutical, cosmetic or cosmeceutical compositions can be used herein, alone or in combination with other propellant. Non-limiting examples of suitable propellants include nitrous oxide, carbon dioxide, nitrogen, and hydrocarbon propellants such as propane, iso-butane, n-butane, isopentane, n-pentane, and dimethyl ether. Preferred propellants are selected from, for example, propane, iso-butane, n-butane, isopentane, n-pentane, and mixtures thereof. Chlorinated fluorocarbons such as 1,1-difluoro- or 1,1,1,2-tetrafluoroethane are also suitable but their use is being limited for environmental reasons. These propellants usually have a low boiling point and are in a gaseous form at room temperature in standard conditions.

The compositions of the present invention can optionally further comprise a variety of components that are suitable for rendering the compositions more cosmetically or aesthetically acceptable or to provide the compositions with additional usage benefits. Such conventional optional components are well known to those skilled in the art and are referred to herein as “ingredients”. These include any cosmetically acceptable ingredients such as those found in the CTFA International Cosmetic Ingredient Dictionary and Handbook, 8th edition, edited by Wenninger and Canterbery, (The Cosmetic, Toiletry, and Fragrance Association, Inc., Washington, D.C., 2000). Some non-limiting representative examples of these ingredients include humectants, deodorants, antiperspirants, sun screening agents, sunless tanning agents, hair conditioning agents, pH adjusting agents, chelating agents, preservatives, emulsifiers, occlusive agents, emollients, thickeners, solubilizing agents, penetration enhancers, anti-irritants, colorants and surfactants.

Thus, the compositions of the present invention can comprise, in combination with ammonium lactate or any other alpha-hydroxy carboxylic acid or a salt thereof, one or more additional humectants or moisturizing agents. Representative examples of humectants that are usable in this context of the present invention include, without limitation, guanidine, glycolic acid and glycolate salts (e.g. ammonium salt and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

The compositions of the present invention can further comprise a pH-adjusting agent. Suitable pH adjusting agents include, for example, one or more adipic acids, glycines, citric acids, calcium hydroxides, magnesium aluminometasilicates, buffers or any combinations thereof.

As is widely recognizable in the art, since the skin pH is 5.5, compositions for topical application should preferably have a pH value of between 4.0 and 7.0, preferably between 5.0 and 6.0, most preferably about 5.5 or substantially 5.5, so as to avoid irritation. Hence, a pH adjusting is typically added so as to bring the PH of the composition to the desired value. The compositions of the present invention are therefore preferably formulated so as to have a pH value that ranges between about 4.0 and about 7.0, more preferably between bout 5.0 and about 6.0.

Representative examples of deodorant agents that are usable in the context of the present invention include, without limitation, quaternary ammonium compounds such as cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmIthyl sarcosine, lauroyl sarcosine, N-myristoyl glycine, potassium N-lauryl sarcosine, stearyl, trimethyl ammonium chloride, sodium aluminum chlorohydroxy lactate, tricetylmethyl ammonium chloride, 2,4,4′-trichloro-2′-hydroxy diphenyl ether, diaminoalkyl amides such as L-lysine hexadecyl amide, heavy metal salts of citrate, salicylate, and piroctose, especially zinc salts, and acids thereof, heavy metal salts of pyrithione, especially zinc pyrithione and zinc phenolsulfate. Other deodorant agents include, without limitation, odor absorbing materials such as carbonate and bicarbonate salts, e.g. as the alkali metal carbonates and bicarbonates, ammonium and tetraalkylammonium carbonates and bicarbonates, especially the sodium and potassium salts, or any combination of the above.

Antiperspirant agents can be incorporated in the compositions of the present invention either in a solubilized or a particulate form and include, for example, aluminum or zirconium astringent salts or complexes.

Representative examples of sun screening agents usable in context of the present invention include, without limitation, p-aminobenzoic acid, salts and derivatives thereof (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one) and 4-isopropyl-di-benzoylmethane, and any combination thereof.

Representative examples of sunless tanning agents usable in context of the present invention include, without limitation, dihydroxyacetone, glyceraldehyde, indoles and their derivatives. The sunless tanning agents can be used in combination with the sunscreen agents.

Suitable hair conditioning agents that can be used in the context of the present invention include, for example, one or more collagens, cationic surfactants, modified silicones, proteins, keratins, dimethicone polyols, quaternary ammonium compounds, halogenated quaternary ammonium compounds, alkoxylated carboxylic acids, alkoxylated alcohols, alkoxylated amides, sorbitan derivatives, esters, polymeric ethers, glyceryl esters, or any combinations thereof.

The chelating agents are optionally added to the compositions of the present invention so as to enhance the preservative or preservative system. Preferred chelating agents are mild agents, such as, for example, ethylenediaminetetraacetic acid (EDTA), EDTA derivatives, or any combination thereof.

Suitable preservatives for use in the compositions of the present composition include, without limitation, one or more alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or any combinations thereof.

Suitable emulsifiers that can be used in the context of the present invention include, for example, one or more sorbitans, alkoxylated fatty alcohols, alkylpolyglycosides, soaps, alkyl sulfates, monoalkyl and dialkyl phosphates, alkyl sulphonates, acyl isothionates, or any combinations thereof.

Suitable occlusive agents that can be used in the context of the present invention include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Suitable emollients, other than ammonium lactate or the alpha-hydroxy carboxylic acid or the salt thereof, that can be used in the context of the present invention include, for example, dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

Suitable thickeners that can be used in the context of the present invention include, for example, non-ionic water-soluble polymers such as hydroxyethylcellulose (commercially available under the Trademark Natrosol^{RTM} 250 or 350), cationic water-soluble polymers such as Polyquat 37 (commercially available under the Trademark Synthalen^{RTM} CN), fatty alcohols, fatty acids and their alkali salts and mixtures thereof.

Representative examples of solubilizing agents that are usable in this context of the present invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEENS and spans, e.g., TWEEN 80. Other solubilizers that are usable for the compositions of the present invention are, for example, polyoxyethylene sorbitan fatty acid ester, polyoxyethylene n-alkyl ethers, n-alkyl amine n-oxides, poloxamers, organic solvents, phospholipids and cyclodextrines.

Suitable penetration enhancers usable in context of the present invention include, but are not limited to, dimethylsulfoxide (DMSO), dimethyl formamide (DMF), allantoin, urazole, N,N-dimethylacetamide (DMA), decylmethylsulfoxide (C₁₀ MSO), polyethylene glycol monolaurate (PEGML), propylene glycol (PG), propylene glycol monolaurate (PGML), glycerol monolaurate (GML), lecithin, the 1-substituted azacycloheptan-2-ones, particularly 1-n-dodecylcyclazacycloheptan-2-one (available under the trademark Azone^{RTM} from Whitby Research Incorporated, Richmond, Va.), alcohols, and the like. The permeation enhancer may also be a vegetable oil. Such oils include, for example, safflower oil, cottonseed oil and corn oil.

Suitable anti-irritants that can be used in the context of the present invention include, for example, steroidal and non steroidal anti-inflammatory agents or other materials such as aloe vera, chamomile, alpha-bisabolol, cola nitida extract, green tea extract, tea tree oil, licoric extract, allantoin, caffeine or other xanthines, glycyrrhizic acid and its derivatives.

The presently known anti-irritants can be divided into water-soluble anti-irritants and water-insoluble anti-irritants. Representative examples of such compositions are described, for example, in U.S. Pat. No. 5,482,710.

In one embodiment of the present invention, compositions of the present invention in which the concentration of ammonium lactate or any other alpha-hydroxy carboxylic acid or salt thereof is higher than 10 weight percentages include one or more anti-irritants.

In another embodiment, the compositions of the present invention, particularly those in which the concentration of ammonium lactate or any other alpha-hydroxy carboxylic acid or salt thereof is lower than 10 weight percentages, do not include an anti-irritant, or include either water-soluble or water-insoluble anti-irritants and are therefore devoid of a combination of these two types of anti-irritants.

Although a wide variety of ingredients can be included in the compositions of the present invention, in addition to the active ingredients, the compositions are preferably devoid of an enduring perfume composition. The incorporation of such a perfume composition in pharmaceutical compositions is considered in the art disadvantageous for skin and scalp medical treatment, as it oftentimes cause undesirable irritation of a sensitive skin.

As used herein, the phrase “an enduring perfume composition” describes a composition that comprises one or more perfumes that provide a long lasting aesthetic benefit with a minimum amount of material. Enduring perfume compositions are substantially deposited and remain on the body throughout any rinse and/or drying steps. Representative examples of such compositions are described, for example, in U.S. Pat. No. 6,086,903.

However, it should be noted that fragrances other than enduring perfume compositions, perfumes or perfume compositions, which are fast removable from the surface they are deposited on, can be included in the compositions of the present invention.

Further optionally, the compositions of the present invention can comprise, in addition to ammonium lactate or any other alpha-hydroxy acid or a salt thereof, one or more other active ingredients (also referred to herein as “additional active ingredient(s)”), which are aimed at providing the composition with an additional therapeutic, cosmeceutical or cosmetic effect.

Hence, as used herein, the phrase “additional active ingredient” refers to an agent, other than ammonium lactate or any other alpha-hydroxy carboxylic acid or a salt thereof, which exert a pharmacological, dermatological or any other beneficial activity.

Compositions according to the present invention, which further comprises one or more additional active ingredients, can therefore be further efficiently used, in addition to treatment of a condition associated with dry skin and/or scalp, in the treatment of any medical, cosmetic and/or cosmeceutical condition in which applying the additional active ingredient is beneficial.

Preferred additional active ingredients according to the present invention include, without limitation, one or more, or any combination of an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an anti-dandruff agent.

Suitable anti-acne agents for use in this context of the present invention include, without limitation, keratolytics such as salicylic acid, sulfur, glycolic, pyruvic acid, resorcinol, and N-acetylcysteine and retinoids such as retinoic acid and its derivatives (e.g., cis and trans, esters).

Suitable antibiotics for use in this context of the present invention include, without limitation, benzoyl peroxide, octopirox, erythromycin, zinc, tetracyclin, triclosan, azelaic acid and its derivatives, phenoxy ethanol and phenoxy proponol, ethylacetate, clindamycin and meclocycline; sebostats such as flavinoids; alpha and beta hydroxy acids; and bile salts such as scymnol sulfate and its derivatives, deoxycholate and cholate.

Representative examples of non-steroidal anti-inflammatory agents that are usable in this context of the present invention include, without limitation, oxicams, such as piroxicam, isoxicam, tenoxicam, sudoxicam, and CP-14,304; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents. For example, etofenamate, a flufenamic acid derivative, is particularly useful for topical application.

Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Suitable antipruritic agents include, without limitation, pharmaceutically acceptable salts of methdilazine and trimeprazine.

Non-limiting examples of anesthetic drugs that are suitable for use in context of the present invention include pharmaceutically acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

Suitable antimicrobial agents, including antibacterial, antifungal, antiprotozoal and antiviral agents, for use in context of the present invention include, without limitation, beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, farnesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, parachlorometa xylenol, nystatin, tolnaftate and clotrimazole and mixtures thereof.

Non-limiting examples of anti-oxidants that are usable in the context of the present invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox^{R}), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulflhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

Non-limiting examples of chemotherapeutic agents usable in context of the present invention include daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A and XR9576.

Non-limiting examples of antidepressants usable in context of the present invention include norepinephrine-reuptake inhibitors (“NRIs”), selective-serotonin-reuptake inhibitors (SSRIs), monoamine-oxidase inhibitors (MAOIs), serotonin-and-noradrenaline-reuptake inhibitors (“SNFIs), corticotropin-releasing factor (CRF) antagonists, α-adrenoreceptor antagonists, NK1-receptor antagonists, 5-HT_{1A}-receptor agonist, antagonists, and partial agonists and atypical antidepressants, as well as norepinephrine-reuptake inhibitors such as, but are not limited to amitriptyline, desmethylamitriptyline, clomipramine, doxepin, imipramine, imipramine-oxide, trimipramine; adinazolam, amiltriptylinoxide, amoxapine, desipramine, maprotiline, nortriptyline, protriptyline, amineptine, butriptyline, demexiptiline, dibenzepin, dimetacrine, dothiepin, fluacizine, iprindole, lofepramine, melitracen, metapramine, norclolipramine, noxiptilin, opipramol, perlapine, pizotyline, propizepine, quinupramine, reboxetine, tianeptine, and serotonin-reuptake inhibitors such as, but are not limited to, binedaline, m-chloropiperzine, citalopram, duloxetine, etoperidone, femoxetine, fluoxetine, fluvoxamine, indalpine, indeloxazine, milnacipran, nefazodone, oxaflazone, paroxetine, prolintane, ritanserin, sertraline, tandospirone, venlafaxine and zimeldine.

Exemplary anti-dandruff ingredients usable in context of the present invention include, without limitation, zinc pyrithione, shale oil and derivatives thereof such as sulfonated shale oil, selenium sulfide, sulfur; salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazolenitrite and any possible stereo isomers and derivatives thereof such as anthralin, piroctone olamine (Octopirox), selenium sulfide, and ciclopirox olamine, and mixtures thereof.

Non-limiting examples of vitamins usable in context of the present invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B₃ (niacinamide and its derivatives), alpha hydroxy acids (such as glycolic acid, lactic acid, tartaric acid, malic acid, citric acid, etc.) and beta hydroxy acids (such as salicylic acid and the like).

Non-limiting examples of dermatological active ingredients usable in context of the present invention include jojoba oil and aromatic oils such as methyl salicylate, wintergreen, peppermint oil, bay oil, eucalyptus oil and citrus oils, as well as ammonium phenolsulfonate, bismuth subgallate, zinc phenolsulfonate and zinc salicylate. Non-limiting examples of antifingal agents include miconazole, clotrimazole, butoconazole, fenticonasole, tioconazole, terconazole, sulconazole, fluconazole, haloprogin, ketonazole, ketoconazole, oxinazole, econazole, itraconazole, terbinafine, nystatin and griseofilvin.

Non-limiting examples of antihistamines usable in context of the present invention include chlorpheniramine, brompheniramine, dexchlorpheniramine, tripolidine, clemastine, diphenhydramine, promethazine, piperazines, piperidines, astemizole, loratadine and terfenadine.

Suitable hormones for use in the context of the present invention include, for example, androgenic compounds and progestin compounds.

Representative examples of androgenic compounds include, without limitation, methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone and pharmaceutically acceptable esters and salts thereof, and combinations of any of the foregoing.

Representative examples of progestin compounds include, without limitation, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5α-pregnan-3β,20α-diol sulfate, 5α-pregnan-3β,20β-diol sulfate, 5α-pregnan-3β-ol-20-one, 16,5α-pregnen-3β-ol-20-one, 4-pregnen-20β-ol-3-one-20-sulfate, acetoxypregnenolone, anagestone acetate, cyproterone, dihydrogesterone, flurogestone acetate, gestadene, hydroxyprogesterone acetate, hydroxymethylprogesterone, hydroxymethyl progesterone acetate, 3-ketodesogestrel, megestrol, melengestrol acetate, norethisterone and mixtures thereof.

The compositions of the present invention may be packed or presented in any convenient way. For example, they may be packed in a tube, a bottle, or a pressurized container, using techniques well known to those skilled in the art and as set forth in reference works such as Remington's Pharmaceutical Science 15^{th} Ed. It is preferred that the packaging is done in such a way so as to minimize contact of the unused compositions with the environment, in order to minimize contamination of the compositions before and after the container is opened.

As the compositions of the present invention include ammonium lactate, or any other alpha-hydroxy carboxylic acid or a salt thereof, preferably in a substantial concentration, these compositions are useful in preventing or treating medical, cosmetic and/or cosmeceutical conditions associated with dry skin and/or scalp such as, for example, xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

Hence, in a preferred embodiment of the present invention, each of the compositions described hereinabove, is packaged in a packaging material and is identified in print, in or on the package, for use in the treatment or prevention of dry skin and/or scalp and/or any one or more of the conditions listed or described herein.

The efficacy of the compositions of the present invention in treating conditions associated with dry skin and scalp is well demonstrated in the Examples section that follows.

Hence, according to another aspect of the present invention, there is provided a method of treating a medical, cosmetic and/or cosmeceutical condition associated with dry skin and/or scalp. The method is effected by topically applying onto the affected biological surface(s) of a subject, e.g., the dry skin and/or scalp, a pharmaceutically, cosmetically or cosmeceutically effective amount of any of the compositions of the present invention as described herein.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The phrase “topically applying” describes application onto one or more biological surface(s), e.g., skin or scalp, by direct laying or spreading a composition on the surface. Non-limiting examples of biological surfaces onto which the compositions of the present invention can be topically applied include the lateral aspect of forearms, the lateral aspect of legs, elbows, palms, feet, backhands, back, scalp and any other dry skin surface.

According to this aspect of the present invention, the compositions of the present invention are preferably topically applied between one and four times a day, more preferably twice a day (e.g., once in the morning and once in the evening). The topical application of the compositions of the present invention is preferably carried out for a time period that ranges between 1 and 30 days, more preferably for a time period of about fourteen days.

The phrase “pharmaceutically, cosmetically or cosmeceutically effective amount” describes an amount of a composition that is sufficient to significantly induce a positive modification in the condition being treated, but low enough to avoid significant side effects, within the scope of sound judgment of the skilled artisan. The effective amount of the composition may vary with the particular skin being treated, the age and physical condition of the biological subject being treated, the severity of the condition, the duration of the treatment, the nature of concurrent therapy, the specific compound, composition or other material employed, the particular pharmaceutically, cosmetically or cosmeceutically acceptable topical carrier utilized, and like factors within the knowledge and expertise of the skilled artisan.

While the method according to this aspect of the present invention is preferably beneficial in treating conditions associated with dry skin and/or scalp, in cases where the compositions of the present invention further comprises additional active ingredient(s), the method can be further used for treating other conditions in which applying the additional active ingredient(s) is beneficial. Such conditions include, for example, infections, fungi, allergies, aging and more.

According to another aspect of the present invention there is provided a process of preparing the novel compositions described hereinabove. The process generally comprises admixing the ammonium lactate or any other alpha-hydroxy carboxylic acid or salt thereof, as described hereinabove, the propellant(s) and the pharmaceutically, cosmetically or cosmeceutically acceptable carrier. In cases were other ingredients or active ingredients, as is detailed hereinabove, are present in the compositions, the process includes admixing these ingredients together with the active ingredients and the carrier. The mixing technique utilized in the process of the present invention can be any one of the known techniques for formulating foamable compositions. A variety of exemplary formulation techniques that are usable in the process of the present invention is described, for example, in Harry's Cosmeticology, Seventh Edition, Edited by J B Wilkinson and RJ Moore, Longmann Scientific & Technical, 1982, Chapter 13 “The Manufacture of Cosmetics” pages 757-799.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Skin and Scalp Compositions

A representative example of a foam skin and scalp topical composition according to the present invention, Composition 1 below, was prepared using conventional methods (see, for example, Harry's Cosmeticology, Seventh Edition, Edited by J B Wilkinson and R J Moore, Longmann Scientific & Technical, 1982, Chapter 13 “The Manufacture of Cosmetics” pages 757-799). The composition comprises about 10 weight percentages of a propellant, as described hereinabove. Other components of the composition are listed hereinbelow.

### Composition 1

| GLYCERINE | 5.0% wt. |
|---|---|
| ALLANTOIN | 0.2% wt. |
| CETYL ALCOHOL | 1.0% wt. |
| POLYSILANE ™ | 3.0% wt. |
| Isopropyl myristate | 4.0% wt. |
| MYRITOL 318 ™ | 3.0% wt. |
| TWEEN 60 ™ | 1.0% wt. |
| SILICON D.C.350 ™ | 0.5% wt. |
| VIT.E ACETATE | 0.1% wt. |
| MONTANOV 68 ™ | 1.5% wt. |
| PHENONIP ™ | 0.7% wt. |
| AMYLUM RICE STARCH ™ | 2.0% wt. |
| PURASAL NH 70 ™* | 17% wt. |
| DMDM HYDANTOIN ™ | 0.35% wt. |
| WATER | qs 100% |
| PH | 5.5 |

*A 70% solution of ammonium lactate

### Example 2

### Comparative Tests

The activity of the composition of the present invention in the treatment of dry skin was tested and compared with that of a control commercially available dry skin product containing ammonium lactate. Thus, a skin foam composition that comprises 12% ammonium lactate (Composition 1 hereinabove) was tested for its activity in the treatment of dry skin according to the protocol described hereinbelow, whereby an Ammonium Lactate 12% cream, marketed by Clay Park Ltd., served as control.

Protocol: The test subjects were healthy volunteers, free of disease, ages 25 to 60. Each volunteer was assigned with a serial number.

Ten volunteers applied the tested preparations on the forearms, as follows: A small amount of the tested preparation was applied to a clean hand. The preparation was rubbed/massaged into the forearm until it was absorbed into the skin. The preparation of this invention was applied on the left forearm. The control preparation was applied on the right forearm.

The participants were asked to complete a questionnaire on their satisfaction with the preparation, once immediately after applying the preparation and a second time after half an hour. The participants were asked to rank the following parameters on a scale from 0 to 5:
Skin roughness/smoothness, after feel, odor, skin irritation, stickiness, skin whitening, absorption, and comfort in use.

The results were graded according to the following scale:
1=bad
2=weak
3=moderate
4=good
5=excellent

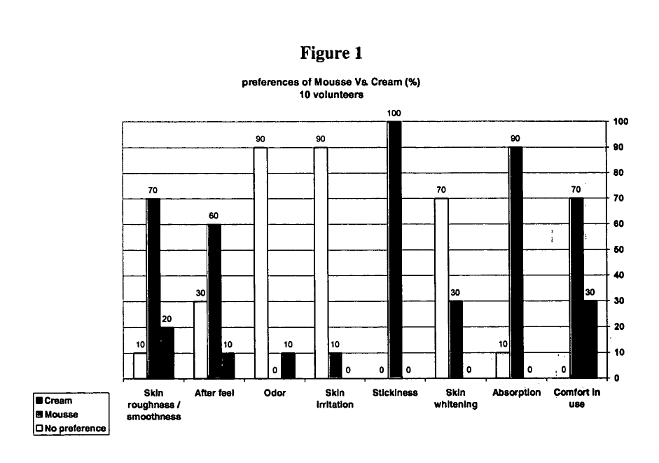

The results from the test are summarized in Table 1 below and are presented in FIG. 1.

The results demonstrate that application of the foam composition of the present invention resulted in advantage in all the examined parameters as compared with application of the commercially available ammonium lactate cream composition.

TABLE 1

| | | | | | | | | |
|---|---|---|---|---|---|---|---|---|
| | Skin | After | | | | | | |
| Volunteer | roughness/smoothness | feel | Odor | Skin irritation | Stickiness | Skin whitening | Absorption | Comfort in use |

COMPOSITION 1 (FOAM)

| | | | | | | | | |
|---|---|---|---|---|---|---|---|---|
| 1 | 3 | 3 | 2 | 5 | 3 | 5 | 4 | 3 |
| 2 | 5 | 5 | 1 | 5 | 5 | 5 | 5 | 5 |
| 3 | 5 | 5 | 5 | 5 | 3 | 5 | 5 | 3 |
| 4 | 5 | 5 | 5 | 5 | 5 | 5 | 5 | 5 |
| 5 | 3 | 3 | 5 | 5 | 4 | 5 | 5 | 4 |
| 6 | 5 | 4 | 5 | 5 | 5 | 5 | 5 | 5 |
| 7 | 5 | 5 | 5 | 5 | 4 | 5 | 5 | 5 |
| 8 | 5 | 5 | 5 | 5 | 5 | 5 | 5 | 5 |
| 9 | 5 | 5 | 5 | 5 | 5 | 5 | 4 | 5 |
| 10 | 5 | 5 | 5 | 5 | 5 | 5 | 5 | 3 |
| Average | 4.6 | 4.5 | 4.3 | 5 | 4.4 | 5 | 4.8 | 4.3 |

CONTROL (CREAM)

| | | | | | | | | |
|---|---|---|---|---|---|---|---|---|
| 1 | 4 | 3 | 5 | 5 | 2 | 5 | 3 | 5 |
| 2 | 3 | 3 | 1 | 5 | 3 | 2 | 2 | 2 |
| 3 | 4 | 5 | 5 | 5 | 1 | 5 | 5 | 5 |
| 4 | 3 | 3 | 5 | 5 | 1 | 5 | 4 | 4 |
| 5 | 5 | 5 | 5 | 5 | 3 | 5 | 2 | 1 |
| 6 | 5 | 3 | 5 | 2 | 3 | 3 | 3 | |
| 7 | 3 | 3 | 5 | 5 | 2 | 5 | 4 | 4 |
| 8 | 3 | 3 | 5 | 5 | 1 | 5 | 3 | 4 |
| 9 | 3 | 4 | 5 | 3 | 1 | 1 | 1 | 3 |
| 10 | 4 | 5 | 5 | 5 | 2 | 5 | 4 | 5 |
| Average | 3.7 | 3.7 | 4.6 | 4.8 | 1.8 | 4.1 | 3.1 | 3.6 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A foamable pharmaceutical, cosmetic or cosmeceutical composition for topical application, identified for use in the treatment of a medical, cosmetic and/or cosmeceutical condition associated with dry skin and/or scalp, comprising ammonium lactate, at least one propellant and a pharmaceutically, cosmetically or cosmeceutically acceptable carrier.

2. The foamable pharmaceutical, cosmetic or cosmeceutical composition of claim 1, packaged in a packaging material and identified in print, in or on said packaging material, for use in the treatment of said condition.

3. The foamable pharmaceutical, cosmetic or cosmeceutical composition of claim 1, wherein said medical, cosmetic and/or cosmeceutical condition is selected from the group consisting of xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

4. The foamable pharmaceutical, cosmetic or cosmeceutical composition of claim 2, wherein said medical, cosmetic and/or cosmeceutical condition is selected from the group consisting of xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

5. The foamable pharmaceutical, cosmetic or cosmeceutical composition of claim 1, wherein a concentration of said ammonium lactate is greater than 5 weight percentages of the composition.

6. The foamable pharmaceutical, cosmetic or cosmeceutical composition of claim 5, wherein said concentration of said ammonium lactate is greater than 10 weight percentages of the composition.

7. The foamable pharmaceutical, cosmetic or cosmeceutical composition of claim 5, wherein said concentration of said ammonium lactate ranges between 5.1 weight percentages and about 30 weight percentages of the composition.

8. The foamable pharmaceutical, cosmetic or cosmeceutical composition of claim 7, wherein said concentration of said ammonium lactate ranges between about 8 weight percentages and about 20 weight percentages of the composition.

9. The foamable pharmaceutical, cosmetic or cosmeceutical composition of claim 1, wherein a concentration of said at least one propellant ranges between about 0.5 weight percentage and about 60 weight percentages.

10. The foamable pharmaceutical, cosmetic or cosmeceutical composition of claim 9, wherein said concentration of said at least one propellant ranges between about 10 weight percentages and about 20 weight percentages.

11. The foamable pharmaceutical, cosmetic or cosmeceutical composition of claim 1, being devoid of an enduring perfume composition.

12. The foamable pharmaceutical, cosmetic or cosmeceutical composition of claim 1, further comprising at least one additional active ingredient.

13. The foamable pharmaceutical, cosmetic or cosmeceutical composition of claim 12, wherein said at least one additional active agent is selected from the group consisting of an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an antidandruff agent.

14. The foamable pharmaceutical, cosmetic or cosmeceutical composition of claim 12, further identified for use in the treatment of a medical, cosmetic and/or cosmeceutical condition in which applying said at least one additional active ingredient is beneficial.

15. The foamable pharmaceutical, cosmetic or cosmeceutical composition of claim 1, further comprising at least one ingredient selected from the group consisting of a humectant, a deodorant agent, an antiperspirant, a sun screening agent, a sunless tanning agent, a hair conditioning agent, a pH adjusting agent, a chelating agent, a preservative, an emulsifier, an occlusive agent, an emollient, a thickener, a solubilizing agent, a penetration enhancer, an anti-irritant, a colorant and a surfactant.

16. The foamable pharmaceutical, cosmetic or cosmeceutical composition of claim 15, wherein said anti-irritant is either a water-soluble anti-irritant or a water-insoluble anti-irritant.

17. The foamable pharmaceutical, cosmetic or cosmeceutical composition of claim 1, having a pH value that ranges between about 4.0 and about 7.0.

18. The foamable pharmaceutical, cosmetic or cosmeceutical composition of claim 17, having a pH value that ranges between about 5.0 and about 6.0.

19. A process of preparing a foamable pharmaceutical, cosmetic or cosmeceutical composition for topical application, identified for use in the treatment of a medical, cosmetic and/or cosmeceutical condition associated with dry skin and/or scalp, the process comprising admixing ammonium lactate, at least one propellant and a pharmaceutically, cosmetically or cosmeceutically acceptable carrier.

20. The process of claim 19, wherein said medical, cosmetic and/or cosmeceutical condition is selected from the group consisting of xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

21. The process of claim 19, wherein a concentration of said ammonium lactate is greater than 5 weight percentages of said composition.

22. The process of claim 21, wherein said concentration of said ammonium lactate is greater than 10 weight percentages of said composition.

23. The process of claim 21, wherein said concentration of said ammonium lactate ranges between 5.1 weight percentages and about 30 weight percentages of said composition.

24. The process of claim 23, wherein said concentration of said ammonium lactate ranges between about 8 weight percentages and about 20 weight percentages of said composition.

25. The process of claim 19, wherein a concentration of said at least one propellant ranges between about 0.5 weight percentage and about 60 weight percentages.

26. The process of claim 25, wherein said concentration of said at least one propellant ranges between about 10 weight percentages and about 20 weight percentages.

27. The process of claim 19, further comprising admixing, with said ammonium lactate, said at least one propellant and said carrier, at least one additional active ingredient.

28. The process of claim 27, wherein said at least one additional active ingredient is selected from the group consisting of an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an antidandruff agent.

29. The process of claim 19, further comprising admixing, with said ammonium lactate, said at least one propellant and said carrier, at least one ingredient selected from the group consisting of a humectant, a deodorant agent, an antiperspirant, a sun screening agent, a sunless tanning agent, a hair conditioning agent, a pH adjusting agent, a chelating agent, a preservative, an emulsifier, an occlusive agent, an emollient, a thickener, a solubilizing agent, a penetration enhancer, an anti-irritant, a colorant, a propellant and a surfactant.

30. The process of claim 29, wherein said anti-irritant is either a water-soluble anti-irritant or a water-insoluble anti-irritant.

31. The process of claim 19, wherein said composition has a pH value that ranges between about 4.0 and about 7.0.

32. The process of claim 31, wherein said composition has a pH value that ranges between about 5.0 and about 6.0.

33. The process of claim 19, wherein said composition is devoid of an enduring perfume composition.

34. A method of treating a medical, cosmetic and/or a cosmeceutical condition, the method comprising topically applying onto at least one biological surface of a subject in need thereof a pharmaceutically, cosmetically or cosmeceutically effective amount of the foamable pharmaceutical, cosmetic or cosmeceutical composition of claim 1.

35. The method of claim 34, wherein said medical, cosmetic and/or cosmeceutical condition is a condition associated with dry skin and/or scalp.

36. The method of claim 35, wherein said condition is selected from the group consisting of xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

37. The method of claim 34, wherein said at least one biological surface is selected from the group consisting of a lateral aspect of a forearm, a lateral aspect of a leg, an elbow, a palm, a foot, a backhand, a back and a scalp.

38. The method of claim 34, wherein a concentration of said ammonium lactate is greater than 5 weight percentages of said composition.

39. The method of claim 38, wherein said concentration of said ammonium lactate is greater than 10 weight percentages of said composition.

40. The method of claim 38, wherein said concentration of said ammonium lactate ranges between 5.1 weight percentages and about 30 weight percentages of said composition.

41. The method of claim 40, wherein said concentration of said ammonium lactate ranges between about 8 weight percentages and about 20 weight percentages of said composition.

42. The method of claim 34, wherein a concentration of said at least one propellant ranges between about 0.5 weight percentage and about 60 weight percentages.

43. The method of claim 42, wherein said concentration of said at least one propellant ranges between about 10 weight percentages and about 20 weight percentages.

44. The method of claim 34, wherein said composition is devoid of an enduring perfume composition.

45. The method of claim 34, wherein said composition further comprises at least one additional active ingredient.

46. The method of claim 45, wherein said at least one additional active ingredient is selected from the group consisting of an antibiotic agent, an antimicrobial agent, an anti-acne agent, an antibacterial agent, an antifungal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an antidandruff agent.

47. The method of claim 45, wherein said medical, cosmetic and/or cosmeceutical condition is a condition in which applying said at least additional active ingredient is beneficial.

48. The method of claim 34, wherein said composition further comprises at least one ingredient selected from the group consisting of a humectant, a deodorant agent, an antiperspirant, a sun screening agent, a sunless tanning agent, a hair conditioning agent, a pH adjusting agent, a chelating agent, a preservative, an emulsifier, an occlusive agent, an emollient, a thickener, a solubilizing agent, a penetration enhancer, an anti-irritant, a colorant, a propellant and a surfactant.

49. The method of claim 48, wherein said anti-irritant is either a water-soluble anti-irritant or a water-insoluble anti-irritant.

50. The method of claim 34, wherein said composition has a pH value that ranges between about 4.0 and about 7.0.

51. The method of claim 50, wherein said composition has a pH value that ranges between about 5.0 and about 6.0.

52. A foamable pharmaceutical, cosmetic or cosmeceutical composition for topical application, identified for use in the treatment of a medical, cosmetic and/or cosmeceutical condition associated with dry skin and/or scalp, comprising at least one alpha-hydroxy carboxylic acid and/or a salt thereof, at least one propellant and a pharmaceutically, cosmetically or cosmeceutically acceptable carrier.

53. The foamable pharmaceutical, cosmetic or cosmeceutical composition of claim 52, packaged in a packaging material and identified in print, in or on said packaging material, for use in the treatment of said condition.

54. The foamable pharmaceutical, cosmetic or cosmeceutical composition of claim 52, wherein said medical, cosmetic and/or cosmeceutical condition is selected from the group consisting of xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

55. The foamable pharmaceutical, cosmetic or cosmeceutical composition of claim 53, wherein said medical, cosmetic and/or cosmeceutical condition is selected from the group consisting of xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

56. The foamable pharmaceutical, cosmetic or cosmeceutical composition of claim 52, wherein said at least one alpha-hydroxy carboxylic acid and/or said salt thereof has a general formula:
RaRbC₁(OH)C₂(═O)OX
wherein:
X is hydrogen, alkyl, cycloalkyl, aryl, halide or an ammonium ion, such that when X is said ammonium ion, said O is negatively charged, or, alternatively, X is a C2-C10 alkyl being attached to said C₂; and
Ra and Rb are each independently hydrogen, halide, alkyl, alkenyl, alkynyl, cycloalkyl or aryl,
or a salt thereof.

57. The foamable pharmaceutical, cosmetic or cosmeceutical composition of claim 56, wherein said at least one alpha-hydroxy carboxylic acid and/or said salt thereof comprises lactic acid and/or a salt thereof.

58. The foamable pharmaceutical, cosmetic or cosmeceutical composition of claim 57, wherein said at least one alpha-hydroxy carboxylic acid and/or said salt thereof comprises ammonium lactate.

59. The foamable pharmaceutical, cosmetic or cosmeceutical composition of claim 52, wherein a concentration of said at least one alpha-hydroxy carboxylic acid and/or said salt thereof is greater than 5 weight percentages of the composition.

60. The foamable pharmaceutical, cosmetic or cosmeceutical composition of claim 59, wherein said concentration of said at least one alpha-hydroxy carboxylic acid and/or said salt thereof is greater than 10 weight percentages of the composition.

61. The foamable pharmaceutical, cosmetic or cosmeceutical composition of claim 59, wherein said concentration of said at least one alpha-hydroxy carboxylic acid and/or said salt thereof ranges between 5.1 weight percentages and about 30 weight percentages of the composition.

62. The foamable pharmaceutical, cosmetic or cosmeceutical composition of claim 61, wherein said concentration of said at least one alpha-hydroxy carboxylic acid and/or said salt thereof ranges between about 8 weight percentages and about 20 weight percentages of the composition.

63. The foamable pharmaceutical, cosmetic or cosmeceutical composition of claim 52, wherein a concentration of said at least one propellant ranges between about 0.5 weight percentage and about 60 weight percentages.

64. The foamable pharmaceutical, cosmetic or cosmeceutical composition of claim 63, wherein said concentration of said at least one propellant ranges between about 10 weight percentages and about 20 weight percentages.

65. The foamable pharmaceutical, cosmetic or cosmeceutical composition of claim 52, being devoid of an enduring perfume composition.

66. The foamable pharmaceutical, cosmetic or cosmeceutical composition of claim 52, further comprising at least one additional active ingredient.

67. The foamable pharmaceutical, cosmetic or cosmeceutical composition of claim 66, wherein said at least one additional active ingredient is selected from the group consisting of an antibiotic agent, an antimicrobial agent, an anti-acne agent, an antibacterial agent, an antifingal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an antidandruff agent.

68. The foamable pharmaceutical, cosmetic or cosmeceutical composition of claim 66, further identified for use in the treatment of a medical, cosmetic and/or cosmeceutical condition in which applying said at least one additional active ingredient is beneficial.

69. The foamable pharmaceutical, cosmetic or cosmeceutical composition of claim 52, further comprising at least one ingredient selected from the group consisting of a humectant, a deodorant agent, an antiperspirant, a sun screening agent, a sunless tanning agent, a hair conditioning agent, a pH adjusting agent, a chelating agent, a preservative, an emulsifier, an occlusive agent, an emollient, a thickener, a solubilizing agent, a penetration enhancer, an anti-irritant, a colorant and a surfactant.

70. The foamable pharmaceutical, cosmetic or cosmeceutical composition of claim 52, wherein said anti-irritant is either a water-soluble anti-irritant or a water-insoluble anti-irritant.

71. The foamable pharmaceutical, cosmetic or cosmeceutical composition of claim 52, having a pH value that ranges between about 4.0 and about 7.0.

72. The foamable pharmaceutical, cosmetic or cosmeceutical composition of claim 71, having a pH value that ranges between about 5.0 and about 6.0.

73. A process of preparing a foamable pharmaceutical, cosmetic or cosmeceutical composition for topical application, identified for use in the treatment of a medical, cosmetic and/or cosmeceutical condition associated with dry skin and/or scalp, the process comprising admixing at least one alpha-hydroxy carboxylic acid and/or said salt thereof, at least one propellant and a pharmaceutically, cosmetically or cosmeceutically acceptable carrier.

74. The process of claim 73, wherein said medical, cosmetic and/or cosmeceutical condition is selected from the group consisting of xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

75. The process of claim 73, wherein said at least one alpha-hydroxy carboxylic acid, said derivative thereof and/or said salt thereof has a general formula:
RaRbC₁(OH)C₂(═O)OX
wherein:
X is hydrogen, alkyl, cycloalkyl, aryl, halide, or an ammonium ion, such that when X is said ammonium ion, said O is negatively charged, or, alternatively, X is a C2-C10 alkyl being attached to said C₂; and
Ra and Rb are each independently hydrogen, halide, alkyl, alkenyl, alkynyl, cycloalkyl or aryl,
or a salt thereof.

76. The process of claim 75, wherein said at least one alpha-hydroxy carboxylic acid and/or said salt thereof comprises lactic acid and/or a salt thereof.

77. The process of claim 76, wherein said at least one alpha-hydroxy carboxylic acid and/or said salt thereof comprises ammonium lactate.

78. The process of claim 73, wherein a concentration of said at least one alpha-hydroxy carboxylic acid and/or said salt thereof is greater than 5 weight percentages of said composition.

79. The process of claim 78, wherein said concentration of said at least one alpha-hydroxy carboxylic acid and/or said salt thereof is greater than 10 weight percentages of said composition.

80. The process of claim 78, wherein said concentration of said at least one alpha-hydroxy carboxylic acid and/or said salt thereof ranges between 5.1 weight percentages and about 30 weight percentages of said composition.

81. The process of claim 80, wherein said concentration of said at least one alpha-hydroxy carboxylic acid and/or said salt thereof ranges between about 8 weight percentages and about 20 weight percentages of said composition.

82. The process of claim 73, wherein a concentration of said at least one propellant ranges between about 0.5 weight percentage and about 60 weight percentages.

83. The process of claim 82, wherein said concentration of said at least one propellant ranges between about 10 weight percentages and about 20 weight percentages.

84. The process of claim 73, further comprising admixing, with said at least one alpha-hydroxy carboxylic acid and/or said salt thereof, said at least one propellant and said carrier, at least one additional active ingredient.

85. The process of claim 84, wherein said additional active ingredient is selected from the group consisting of an antibiotic agent, an antimicrobial agent, an anti-acne agent, an antibacterial agent, an antifingal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an antidandruff agent.

86. The process of claim 73, further comprising admixing, with said at least one alpha-hydroxy carboxylic acid and/or said salt thereof, said at least one propellant and said carrier, at least one ingredient selected from the group consisting of a humectant, a deodorant agent, an antiperspirant, a sun screening agent, a sunless tanning agent, a hair conditioning agent, a pH adjusting agent, a chelating agent, a preservative, an emulsifier, an occlusive agent, an emollient, a thickener, a solubilizing agent, a penetration enhancer, an anti-irritant, a colorant, a propellant and a surfactant.

87. The process of claim 73, wherein said anti-irritant is either a water-soluble anti-irritant or a water-insoluble anti-irritant.

88. The process of claim 73, wherein said composition has a pH value that ranges between about 4.0 and about 7.0.

89. The process of claim 88, wherein said composition has a pH value that ranges between about 5.0 and about 6.0.

90. The process of claim 73, wherein said composition is devoid of an enduring perfume composition.

91. A method of treating a medical, cosmetic and/or a cosmeceutical condition, the method comprising topically applying onto at least one biological surface of a subject in need thereof a pharmaceutically, cosmetically or cosmeceutically effective amount of the foamable pharmaceutical, cosmetic or cosmeceutical composition of claim 52.

92. The method of claim 91, wherein said medical, cosmetic and/or cosmeceutical condition is a condition associated with dry skin and/or scalp.

93. The method of claim 92, wherein said condition is selected from the group consisting of xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, warts, dandruffs and xeroderma pigmentosum.

94. The method of claim 91, wherein said at least one biological surface is selected from the group consisting of a lateral aspect of a forearm, a lateral aspect of a leg, an elbow, a palm, a foot, a backhand, a back and a scalp.

95. The method of claim 91, wherein said at least one alpha-hydroxy carboxylic acid and/or said salt thereof has a general formula:
RaRbC₁(OH)C₂(═O)OX
wherein:
X is hydrogen, alkyl, cycloalkyl, aryl, halide, or an ammonium ion, such that when X is said ammonium ion, said O is negatively charged, or, alternatively, X is a C2-C10 alkyl being attached to said C₂; and
Ra and Rb are each independently hydrogen, halide, alkyl, alkenyl, alkynyl, cycloalkyl or aryl,
or a salt thereof.

96. The method of claim 95, wherein said at least one alpha-hydroxy carboxylic acid and/or said salt thereof comprises lactic acid and/or a salt thereof.

97. The method of claim 96, wherein said at least one alpha-hydroxy carboxylic acid and/or said salt thereof comprises ammonium lactate.

98. The method of claim 91, wherein a concentration of said at least one alpha-hydroxy carboxylic acid and/or said salt thereof is greater than 5 weight percentages of said composition.

99. The method of claim 98, wherein said concentration of said at least one alpha-hydroxy carboxylic acid and/or said salt thereof is greater than 10 weight percentages of said composition.

100. The method of claim 98, wherein said concentration of said at least one alpha-hydroxy carboxylic acid and/or said salt thereof ranges between 5.1 weight percentages and about 30 weight percentages of said composition.

101. The method of claim 100, wherein said concentration of said at least one alpha-hydroxy carboxylic acid and/or said salt thereof ranges between about 8 weight percentages and about 20 weight percentages of said composition.

102. The method of claim 91, wherein a concentration of said at least one propellant ranges between about 0.5 weight percentage and about 60 weight percentages.

103. The method of claim 102, wherein said concentration of said at least one propellant ranges between about 10 weight percentages and about 20 weight percentages.

104. The method of claim 91, wherein said composition is devoid of an enduring perfume composition.

105. The method of claim 91, wherein said composition further comprises at least one additional active ingredient.

106. The method of claim 105, wherein said at least one additional active ingredient is selected from the group consisting of an antibiotic agent, an antimicrobial agent, an anti-acne agent, an antibacterial agent, an antifingal agent, an antiviral agent, a steroidal anti-inflammatory agent, a non-steroidal anti-inflammatory agent, an anesthetic agent, an antipruriginous agent, an antiprotozoal agent, an anti-oxidant, a chemotherapeutic agent, an antidepressant, an anti histamine, a vitamin, a hormone and an antidandruff agent.

107. The method of claim 105, wherein said medical, cosmetic and/or cosmeceutical condition is a condition in which applying said at least additional active ingredient is beneficial.

108. The method of claim 91, wherein said composition further comprises at least one ingredient selected from the group consisting of a humectant, a deodorant agent, an antiperspirant, a sun screening agent, a sunless tanning agent, a hair conditioning agent, a pH adjusting agent, a chelating agent, a preservative, an emulsifier, an occlusive agent, an emollient, a thickener, a solubilizing agent, a penetration enhancer, an anti-irritant, a colorant, a propellant and a surfactant.

109. The method of claim 108, wherein said anti-irritant is either a water-soluble anti-irritant or a water-insoluble anti-irritant.

110. The method of claim 91, wherein said composition has a pH value that ranges between about 4.0 and about 7.0.

111. The method of claim 110, wherein said composition has a pH value that ranges between about 5.0 and about 6.0.
